(19) United States
(12) Patent Application Publication
(10) Pub. No.: US 2015/0157754 A1
(43) Pub. Date: Jun. 11, 2015

(54) DISPENSERS FOR, AND METHODS OF, DISINFECTING HANDS

(71) Applicants: Joseph Daniel Rutter, Independence, OH (US); David Richard Macinga, Stow, OH (US); Nick E. Ciavarella, Seven Hills, OH (US)

(72) Inventors: Joseph Daniel Rutter, Independence, OH (US); David Richard Macinga, Stow, OH (US); Nick E. Ciavarella, Seven Hills, OH (US)

(73) Assignee: GOJO INDUSTRIES, INC., Akron, OH (US)

(21) Appl. No.: 14/549,733

(22) Filed: Nov. 21, 2014

Related U.S. Application Data
(60) Provisional application No. 61/914,774, filed on Dec. 11, 2013.

(51) Int. Cl. A61L 2/24; A61L 2/18; A47K 5/12

(52) U.S. Cl. CPC A61L 2/24; A47K 5/1217; A61L 2/18

(57) ABSTRACT

Exemplary dispensers for providing fresh active ingredients to a user's hands over a period of time and exemplary methods of disinfecting hands are disclosed herein. In one exemplary embodiment, the dispenser includes a sensor for sensing the presence of one or more hands, a controller is in circuit communication with the sensor. A container for holding an oxidizer is also included as well as a pump for pumping the oxidizer out of the container. The dispenser includes logic for controlling the pump and an outlet for dispensing the oxidizer. Upon detection of one or more hands, the logic causes the pump to dispense the oxidizer over a period of time that is greater than 20 seconds.

### RELATED APPLICATIONS

This application claims priority to and the benefits of U.S. Provisional Patent Application Ser. No. 61/914,774 filed on Dec. 11, 2013 and entitled “DISPENSERS FOR, AND METHODS OF, DISINFECTING HANDS,” which is incorporated herein by reference in its entirety.

### TECHNICAL FIELD

The present invention relates generally to liquid dispenser systems and more particularly to dispenser systems that provide a continuous stream of oxidizers or antimicrobial compounds to a user's hands and methods of disinfecting hands.

### BACKGROUND OF THE INVENTION

Oxidizers and other protein sensitive antimicrobials kill microorganisms very effectively in test tubes and on surfaces, however, it is believed that they do not kill microorganisms very well on a person's hands or skin because the active ingredients in these antimicrobials react with the skin and are quickly used up. To compensate for this, such antimicrobials are often used at high concentrations which irritate and/or dry out the skin. Another method of compensating for the reaction of the active ingredients with the skin is to soak a user's hands in a bath containing antimicrobials at a lower concentration. A need using oxidizers and protein sensitive antimicrobial compounds at lower concentrations without having to soak ones hands in a large baths is needed.

### SUMMARY

Exemplary dispensers for providing fresh active ingredients to a user's hands over a period of time and exemplary methods of disinfecting hands are disclosed herein. In one exemplary embodiment, the dispenser includes a sensor for sensing the presence of one or more hands, a controller is in circuit communication with the sensor. A container for holding an oxidizer is also included as well as a pump for pumping the oxidizer out of the container. The dispenser includes logic for controlling the pump and an outlet for dispensing the oxidizer. Upon detection of one or more hands, the logic causes the pump to dispense the oxidizer over a period of time that is greater than about 10 seconds.

Another exemplary dispenser for providing fresh active ingredients to a user's hands over a period of time includes a sensor for sensing the presence of one or more hands, a controller in circuit communication with the sensor, a container for holding an antimicrobial compound, a pump for pumping the antimicrobial compound out of the container, logic for controlling the operation of the pump and an outlet for dispensing the antimicrobial compound. Upon detection of one or more hands, the controller causes the pump to dispense a continuous stream of antimicrobial compound over a period of time that is greater than 20 seconds.

Another exemplary dispenser for providing fresh active ingredients to a user's hands over a period of time includes a sensor for sensing the presence of one or more hands, a controller in circuit communication with the sensor, a container for holding a fluid, a pump for pumping the fluid out of the container, logic for controlling the operation of the pump and an outlet for dispensing the fluid. Upon detection of one or more hands, the logic causes the pump to dispense a continuous stream of fluid over a period of time that is greater than 20 seconds.

An exemplary method of disinfecting hands with an oxidizer includes providing a dispenser that dispenses an oxidizer over a period of time that is greater than about 30 seconds, providing an oxidizer for the dispenser; and inducing a user to rub their hands together under an outlet of the dispenser for the period of time.

A method of killing C-Diff includes providing a container for holding an oxidixer, outputting a stream of oxidizer onto one or more hands for a period of time that is greater than about 10 seconds and instructing a user to rub her hands in the stream of oxidizer.

### BRIEF DESCRIPTION OF THE DRAWINGS

These and other features and advantages of the present invention will become better understood with regard to the following description and accompanying drawings in which:

FIG. 1 is a schematic view of an exemplary embodiment of a dispenser system; and

FIGS. 2 and 3 are block diagrams of exemplary logic for controlling a dispenser.

### DETAILED DESCRIPTION

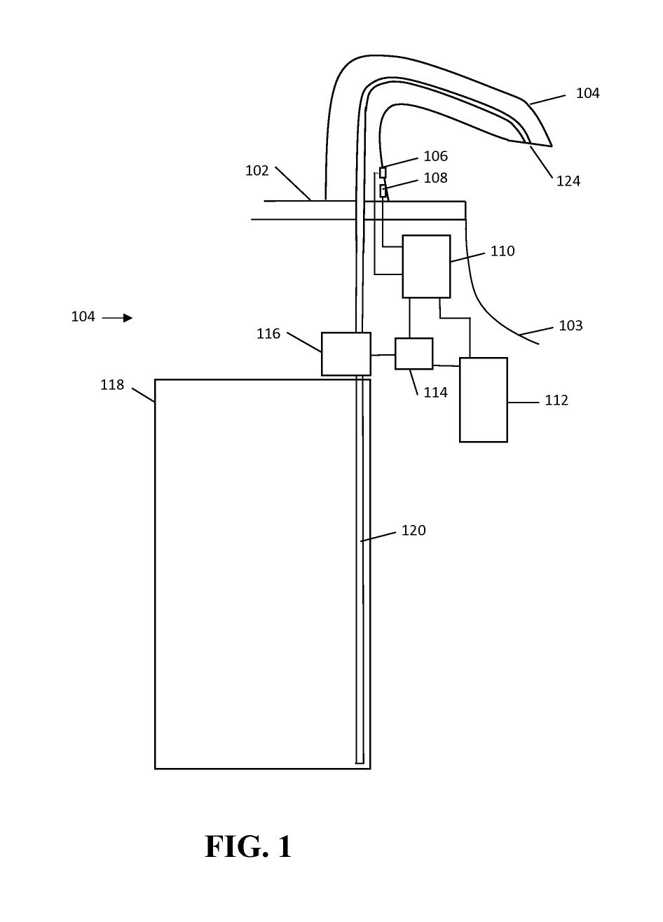

FIG. 1 is a schematic view of an exemplary embodiment of a dispenser system 100. Dispenser system 100 is a counter-mount dispenser system, however, any type of dispenser may be used, such as, for example, a wall mounted dispenser, a stand mounted dispenser, a standalone dispenser, or the like. Dispenser 100 includes a spout 104, which is mounted to a countertop 102. Spout 104 includes an object sensor 196, such as, for example, an infrared sensor, a motion sensor, a capacitance sensor or the like. Sensor 106 is in circuit communication with controller 110. Controller 110 may include a processor, a microprocessor or the like. Controller 110 also includes any necessary memory or circuitry required to perform the functions described herein. In addition, spout 104 includes feedback indicator 108. Feedback indicator 108 may provide a visual and/or an audible feedback to a user. Exemplary visual feedback indicators maybe, for example, one or more light emitting diodes (LEDs). In addition, controller 110 is in circuit communication with pump drive 114.

“Circuit communication” indicates a communicative relationship between devices. Direct electrical, electromagnetic and optical connections and indirect electrical, electromagnetic and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers or satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices not directly connected to each other, but both capable of interfacing with a third device, such as, for example, a CPU, are in circuit communication.

A power source 112 provides power to the controller 110, pump drive 114 and any other components that require power. Power supply 112 may be one or more batteries, or may be a hard wired power source and draw power, from for example, an 120 VAC line. In such case, power supply 112 may include any necessary transformers, rectifiers, or power conditioning devices to obtain suitable power for the components described herein. Pump drive 114 drives pump 116.

Pump 116 is connected to inlet dip tube 120, which is located in reservoir 118 and an outlet tube 122 that extends up through spout 104 to outlet 124. Pump 116 as described herein is a liquid pump; however, in some embodiments, pump 116 is a foam pump. Accordingly, the fluid may be dispensed as a liquid or as a foam.

Reservoir 118 contains a fluid, such as, for example, an oxidizer. Exemplary oxidizers include, for example, peroxygen compounds, hydrogen peroxide, peroxypyruvic acid, peracetic acid, bleach, chlorine compounds, permaganates, sanitizers and the like. In some embodiments the fluid is an antimicrobial, such as, for example, a quaternary ammonium compound, an iodine compound, chlorhexidine, or the like. In some embodiments, the oxidizers may be diluted from a concentrate just prior to being dispensed. In some embodiments, the oxidizers are diluted with water in rations from between about 1:100 to about 1:1000.

Controller 110 includes logic or circuitry for operating pump 116. “Logic” is synonymous with “circuit” or “circuitry” and includes, but is not limited to hardware, firmware, software and/or combinations of each to perform a function(s) or an action(s). For example, based on a desired application or needs, logic may include a software controlled microprocessor or microcontroller, discrete logic, such as an application specific integrated circuit (ASIC) or other programmed logic device. Logic may also be fully embodied as software. The circuits identified and described herein may have many different configurations to perform the desired functions.

Also, voltages and values representing digitized voltages are considered to be equivalent for the purposes of this application, and thus the term “voltage” as used herein refers to either a signal, or a value in a processor representing a signal, or a value in a processor determined from a value representing a signal.

“Signal,” includes, but is not limited to one or more electrical signals, analog or digital signals, one or more computer instructions, a bit or bit stream, or the like.

Any values identified in the detailed description are exemplary and they are determined as needed for a particular dispenser and/or refill design. Accordingly, the inventive concepts disclosed and claimed herein are not limited to the particular values or ranges of values used to describe the embodiments disclosed herein.

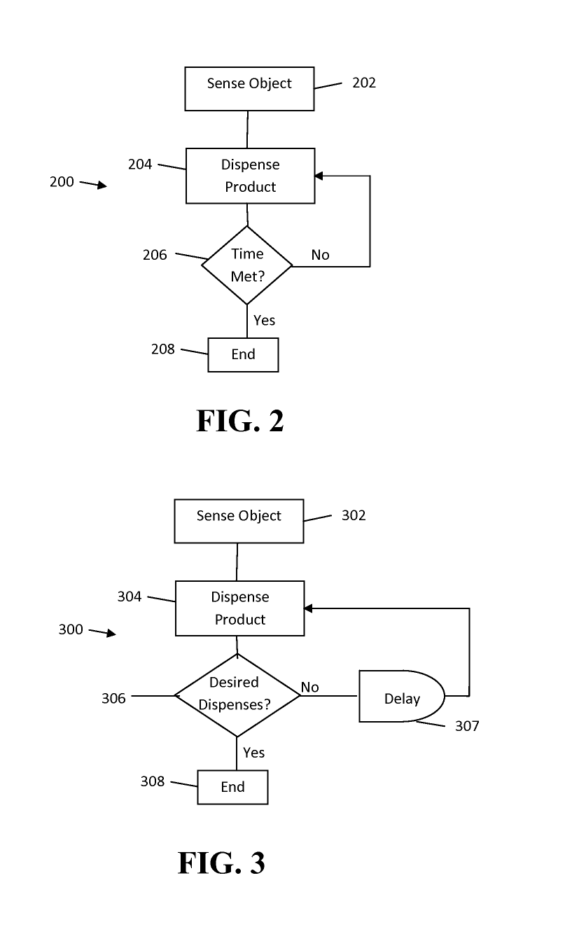

FIG. 2 illustrates exemplary logic 200 for control circuitry 110 for dispensing a continuous stream of fluid on skin. In some embodiments the fluid is an oxidizer. In some embodiments the fluid is an oxidizer diluted with water. The exemplary logic 200 detects the presence of an object at block 202. At block 204 dispenser 100 begins dispensing product. A determination is made at block 206 as to whether a time limit has been reached. The time limit may be, for example, 10 seconds, 20 second, 30 seconds, 60 seconds or the like. If the time limit has not been met, the logic loops back to block 204 and continues to dispense fluid. If the time limit has been met, the logic proceeds to block 208 and the dispensing of fluid ends.

The time limits may be predetermined time limits. The time limits may be a variable time limit. In some embodiments, the time limit may be adjustable in, for example, the field to accommodate different types of locations that require more or less stringent disinfection routines.

FIG. 3 illustrates another exemplary logic 300 for control circuitry 110 for intermittently dispensing a stream of fluid on skin. An object is detected at block 302. At block 304 a discrete shot of fluid is dispensed. At block 306 a determination is made as to whether a desired amount of dispenses have been made. If the desired amount of dispenses has not been made at block 306 the logic proceeds to block 307. At block 307 logic 300 is delayed. The delay may be for a set period of time. The period of time may be preset or may be variable. After the delay period expires, logic 300 flows back to block 304 and dispenses a shot of fluid. In addition, the delay may be a function of the number of dispenses previously made. In addition, in some embodiments, a number of dispenses is selected and the delay is a function of the number of dispenses selected. If at block 306 the desired number of dispenses have been dispensed, the logic ends at block 308.

In some embodiments, block 306 determines whether a set time limit has passed rather than a number of dispenses. In some embodiments, an audible or visual indicator provides a signal to the user prior to dispensing fluid. In some embodiments, an audible of visual indicator provides a signal to the user that she has washed her hands for the required time limit. In some embodiments, the object sensor detects movement of the hands and a signal is provided to the user that she needs to move her hands. In addition, one or more of these actions may be incorporated in whole or in part with the logic described herein for operating a dispenser.

Typical hand washing involves applying a single dose of fluid to ones hands and rubbing them together for a period of time. It has been discovered that applying a single dose of fluid (or even multiple doses of fluid at the same time) to ones hands and rubbing them together does not have a very good efficacy in microbial kill rates. For example, in one experiment, a 10 ml dose of peroxypyruvic acid (“PPA”) was applied to users' hands and the users rubbed their hands together for 60 seconds. These users averaged about a 1.7 log reduction in the Clostridium difficile spores.

However, it has been found that applying multiple doses of fluid over a period of time, or applying a continuous stream of fluid over a period of time increases the efficacy of oxidizers and antimicrobial compounds even though with these methods the same amount of fluid is used for the same amount of time as a typical hand washing event.

Experiments were conducted by contaminating fingertips with 0.005 mL of a 9.0 log cfu/ml bacterial suspension and opposite fingers were rubbed together until dry. Product was dispensed from a syringe and rubbed over all surfaces of both hands so that hands remain wet throughout the hand disinfection procedure and dripping was minimized. Bacteria were recovered by rubbing the fingertips in a petri dish containing sampling fluid for 1 minute.

For the standard treatment, all of the product was applied to the hands in the first few seconds of the hand disinfection procedure. For the continuous treatment, the product was added in several small aliquots over the course of the hand disinfection procedure. The volume of product and the exposure time is the same for both treatments.

In one experiment, a 5 ml dose of PPA was applied to users' hands and the user rubbed their hands together for 30 seconds and a second 5 ml dose of PPA was applied to the users' hands and the users continued to rub their hands for 30 more seconds. The second experiment resulted in a log reduction of about 3.9. Thus, the experiments resulted in an increase of about 2.2 log reduction in the kill rate.

Another experiment involved users receiving a dose of 0.01% bleach and rubbing their hands together of 20 seconds. A single dose of 0.01% bleach resulted in less than about a 1.5 log reduction in microorganisms. Next, a very small continuous stream of 0.01% bleach was applied to the users' hands while they rubbed their hands together for 20 seconds. This resulted in about a 2.1 log reduction in the microorganisms. Thus, continuous application of an oxidizer over a set hand rubbing time resulted in a higher kill rate than a single dose applied at the beginning of the hand rubbing time.

Other experiments involved applying 0.005 ml of 9 log CFU/ml Clostridium difficile (“C-Diff”) ATCC 700057 spore suspension to one or more fingerpads and rubbing opposite fingers together until dry. Contaminated fingerpads underwent a water rinse treatment, a bleach rub treatment or a bleach rinse treatment.

For the water rinse treatment, opposite fingerpads were rubbed together under a 0.01 M phosphate buffer pH 6.5 stream from a wash bottle for 30 seconds. The remaining spores were recovered by rubbing individual fingerpads on the bottom of a petri dish containing 5 ml neutralizer. The spores were plated in duplicate on brain heart infusion agar with sodium taurocholate and incubated anaerobically for 48 hours. The log reductions were calculated by subtracting the mean log10 CFU that was recovered following treatment from the mean log10 CFU recovered from an untreated finger. The water rinse treatment resulted in about a 1 log reduction in the C-Diff spores.

For the breach rub treatment, a 0.02 ml of 1:100 dilution of bleach in 0.01 M phosphate buffer pH 6.5 (the “product”) was applied to one fingerpad and rubbed with opposite fingerpad for 15 seconds. Another 0.02 ml of product was applied to one fingerpad after 15 seconds to make sure fingers remained wet with product the entire time. The remaining spores were recovered by rubbing individual fingerpads on the bottom of a petri dish containing 5 ml neutralizer. The spores were plated in duplicate on brain heart infusion agar with sodium taurocholate and incubated anaerobically for 48 hrs. The log reductions were calculated by subtracting the mean log10 CFU that was recovered following treatment from the mean log10 CFU recovered from an untreated finger. The bleach rub treatment resulted in about a 0.2 log reduction in the C-Diff spores.

For the Bleach rinse treatment, opposite fingerpads were rubbed together under a product stream from a wash bottle for 30 seconds. The remaining spores were recovered by rubbing individual fingerpads on the bottom of a petri dish containing 5 ml neutralizer. The spores were plated in duplicate on brain heart infusion agar with sodium taurocholate and incubated anaerobically for 48 hrs. The log reductions were calculated by subtracting the mean log10 CFU that was recovered following treatment from the mean log10 CFU recovered from an untreated finger. The bleach rinse treatment resulted in about a 2.7 log reduction in the C-Diff spores.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and illustrative examples shown and described. Accordingly, departures may be made from such details without departing from the spirit or scope of the applicant's general inventive concept.

We claim:

1. A touch free dispenser for providing fresh active ingredients to a user's hands over a period of time comprising:
a sensor for sensing the presence of one or more hands;
a controller in circuit communication with the sensor;
a container for holding an oxidizer;
a pump for pumping the oxidizer out of the container;
logic for controlling the pump;
an outlet for dispensing the oxidizer;
wherein upon detection of one or more hands, the controller causes the pump to dispense the oxidizer over a period of time that is greater than about 10 seconds.

2. The touch-free dispenser of claim 1 wherein the dispenser dispenses a continuous stream of the oxidizer.

3. The touch-free dispenser of claim 1 wherein the dispenser dispenses a continuous mist of the oxidizer.

4. The touch-free dispenser of claim 1 wherein the dispenser dispenses multiple shots of the oxidizer at predetermined time intervals.

5. The touch-free dispenser of claim 1 wherein the dispenser dispenses intermittent doses of the oxidizer.

6. The touch-free dispenser of claim 5 wherein the intermittent doses of the oxidizer are dispensed at predetermined time intervals.

7. The touch-free dispenser of claim 1 wherein the oxidizer includes one or more of peroxypyruvic acid, chlorine, a peroxygen compound, hydrogen peroxide, peracetic acid or a permanganate.

8. The touch-free dispenser of claim 1 wherein the period of time is greater than about 30 seconds.

9. The touch-free dispenser of claim 1 wherein the object sensor further detects movement of the hands and when the movement drops below a threshold level and one or more hands are present, the dispenser dispenses a shot of oxidizer.

10. The touch-free dispenser of claim 1 further comprising a sensory feedback indicator for indicating a period of time.

11. The touch-free dispenser of claim 1 wherein the oxidizer dispensed over the period of time is less than about 20 milliliters of oxidizer.

12. The touch-free dispenser of claim 1 wherein the oxidizer dispensed over the period of time is less than about 15 milliliters of oxidizer.

13. The touch-free dispenser of claim 1 wherein the oxidizer dispensed over the period of time is less than about 10 milliliters of oxidizer.

14. A dispenser for providing fresh active ingredients to a user's hands over a period of time comprising:
a sensor for sensing the presence of one or more hands;
a controller in circuit communication with the sensor;
a container for holding an antimicrobial compound;
a pump for pumping the antimicrobial compound out of the container;
logic for controlling the operation of the pump;
an outlet for dispensing the antimicrobial compound;
wherein upon detection of one or more hands, the controller causes the pump to dispense a continuous stream of antimicrobial compound over a period of time that is greater than about 10 seconds.

15. The touch-free dispenser of claim 14 wherein the fluid includes more than one antimicrobial compounds.

16. The touch-free dispenser of claim 15 wherein the antimicrobial compound includes one or more of quaternary ammonium compounds, iodine compounds or chlorhexidine compounds.

17. A dispenser for providing fresh active ingredients to a user's hands over a period of time comprising:
a sensor for sensing the presence of one or more hands;
a controller in circuit communication with the sensor;
a container for holding a fluid;
a pump for pumping the fluid out of the container;
logic for controlling the operation of the pump;
an outlet for dispensing the fluid;
wherein upon detection of one or more hands, the logic causes the pump to dispense a continuous stream of fluid over a period of time that is greater than about 10 seconds.

18. A method of killing C-Diff comprising:
providing a container for holding an oxidixer;
outputting a stream of oxidizer onto one or more hands for a period of time that is greater than 20 seconds;
instructing a user to rub her hands in the stream of oxidizer.

19. The method of claim 18 wherein the stream of oxidizer is an intermittent stream.

20. The method of claim 18 wherein the oxidizer comprises a bleach.
